(19) United States
(12) Patent Application Publication
(10) Pub. No.: US 2004/0071790 A1
(43) Pub. Date: Apr. 15, 2004

(54) Ayurvedic formulation for treating acquired immune deficiency syndrome (HIV/AIDS) patients to obtain negative results of HIV/AIDS infection & other dreaded diseases and process for preparing the same

(76) Inventor: Poturaju Surya Venkata Satyanarayana, Hyderabad (IN)

Correspondence Address: POTURAJU SURYA VENKATA SATYANARAYANA, # 1-8-499/6, Road No. 9, Venkateshwara Swamy Temple Street, Chikkadpally, HYDERABAD, AP, 500020, IN

(21) Appl. No.: 10/266,785

(22) Filed: Oct. 9, 2002

(51) Int. Cl. A61K 35/78

(52) U.S. Cl. 424/725

(57) ABSTRACT

Ayurvedic combination of medicines for treatment/cure of HIV/AIDS patients with Anubhuta Yoga includes therapeutically effective doses of four combinations of medicines. First combination therapy for first two months three types of capsules per day (2+2+1=5) total 5.—its contents include composition—I plus composition—III combined twice daily upto 1^{st} and 2^{nd} month, composition—II per day only 1 cap once upto four months. After completion of 2 months second combination therapy for 2 months includes three types of capsules per day 2+2+1 total 5—its contents include in the place of composition—I given composition—IV plus composition—III combined per day twice (3^{rd} and 4^{th} month) and composition—II per day only once as earlier taken. Schedule of therapy is carried on for Adults only with certain diet and other restrictions.

### CROSS-REFERENCE TO RELATED APPLICATION

This is a completely different application of USPTO earlier published application Ser. No. 09/905,597, filed Jul. 12, 2001 and which contents of difference are stated below.

With the usage of these medicines, the person can become normal as before. There is no age bar i.e. from child to old age. (Pregnant and Lactating Women also). There are no side effects at all. There is no need for the patient to stay in the hospital as inpatient, he can stay at home and get treatment done. Treatment is given according to the age and day-by-day improvement is observed.

If the patient is having any associated diseases, can use allopathic treatment simultaneously, staying at home until those associated symptoms/diseases subside, and use this medicine for the main disease. Patient can use allopathic drug along-with this medicine, as these drugs are not contra-indicated or any side effects with any allopathic medicine.

The same drugs are being tried in HIV complicated cases with:—

(1) T.B. by T.B. & Chest Specialist.

(2) Dermatological Disorders by Dermatologist.

(3) Cancer—Malignant Diseases by Oncologist.

(4) Psychiatric Disorders by Neuro-Physchiatrist.

They found remarkable improvement clinical, radiological and bacteriological aspects of their patients without any side effects. With these combination of medicines patient can know his health condition through any Diagnostic tests i.e. Elisa test, Western Blot test, HIV RNA PCR ULTRA QUANT (Viral Load) and Absolute CD4 count.

These medicines combat successfully the “RETROVIRUS” in the body system of the patient during and after treatment producing inherent improvement in the CD4 and CD8 cells status with satisfactory reduction of PCR (infections particles per ml). That is “viral load” is reduced to normal condition from 4 months onwards and from 1 year to 3 years. During this period no more anti HIV/AIDS medicines will be administered again as re-treatment is not required when patient takes precautions. Duration of Treatment is 4 Months Only.

To know the efficiency of the drug 15 days is sufficient, as Body Heat/feverishness subsides within 24 to 48 hours. It means drug action starts to destroy/reduction of virus within minutes and to normalize the organs to function.

Today in the world, in any system of medicine, virus cannot be destroyed/killed, but with this combination of medicine it is proved practically that virus can be destroyed/killed without any toxicity to the cells.

All organs come to normal as before with the usage of this medicine as virus is destroyed/killed. With the usage of this medicine absolute CD4 count of all the patients becomes normal/increase.

In these cases actually we are saying HIV comes to Negative within 1 to 3 years, depending upon Viral present in the body. But we are giving the medicine only for 4 months with the action of medicine and also good confidence in the patient.

If the patients follow the precautions i.e. with regard to diet, sex, less exposure to sun, working late night etc then the immunity in the body develops to such an extent day by day viral destroyed and CD 4 raised with this medicine.

The action of the drugs used will be more even after usage of medicine, provided if the patient follows the precautions after completion of course. Hence 4 months course is sufficient to treat patient stated as before.

VERSATILITY—These drugs can be used along with allopathic drugs without any drug interaction to all age groups. Cure rate -99% if the patient if not having any associated disease. Cure rate also depends on the patients' follow-up of the restrictions given by us.

The drugs are tried in-patients—declared positive through ELISA, WESTERN BLOT & VIRAL LOAD tests where CD4 absolute count is decreased. By using these drugs CD4 absolute count comes back to normal within 4 to 6 months. But the clinical improvement is noticed from 48 hours after the commencement of treatment & within 15 days treatment, 30% of symptoms subside.

The patients are given treatment for a period of 4 months. For any kind of associated disease like T.B., Cancer, Dermatological problems, Psychological Problems, they are referred to Specialist Doctors in the respective fields.

We have also given below detailed explanation of the efficacy of Ayurvedic medicines in the treatment/cure of HIV/AIDS. Proved by Diagnostics Methods i.e. Elisa, Western Blot, Viral Load, Absolute CD4 tests.

The inventor has proved treatment/cure in Allopathy exclusion criteria also with treated HIV/AIDS patients. Successfully without any side effects. i.e., Past history of convulsions or cardiac diseases and currently requiring medication for control, those having taken AZT in the immediately preceding 4 weeks, Pregnant and lactating women. For patients with associated diseases—T.B., Dermatological Disorders, Cancer & Psychiatric Disorders the Inventor while continuing his treatment has referred them to T.B. & Chest Specialist, Dermatologist, Oncologist and Neuro-Psychiatrist, where they found remarkable improvement in clinical, radiological and bacteriological aspects of their patients.

### STATEMENT REGARDING FEDERALLY SPONSORED RESEARCH OR DEVELOPMENT

“Not Applicable”

### REFERENCE TO SEQUENCE LISTING, A TABLE, OR A COMPUTER PROGRAM LISTING COMPACT DISK APPENDIX

“Not Applicable”

### BACKGROUND OF THE INVENTION

This invention pertains to herbal (Ayurvedic), formulations and method for preparing the Ayurvedic formulations which can be used for treating AIDS and immune-deficient patients suffering with Cancer and Tuberculosis to obtain negative results of HIV/AIDS infectious patients. Simultaneously Allopathic medicines can be used for other diseases like T.B, Cancer etc.

### DESCRIPTION OF THE BACKGROUND ART

In ancient periods of Indian culture Sushruta Charya used the term “VYAVAYA SOSHA” while narrating the evil effects (Symptoms) of perverted Sex indulgence. The same term is also used while recommending the line of treatment. It is interesting to note ‘SOSHA’ (name of the disease syndrome) as one of the communicable disease i.e. spreading from one to another person (Samkramita diseases described in Ayurvedic classics).

In this context as the present scenario of the disease so-called AIDS, which is a global burning problem is said to be discovered/invented in the present trend of life.

The present invention describes formulation and process of making botanical (Ayurvedic) formulation for treating the dreaded disease HIV/AIDS in humans and to bring the CD4 count to the normal levels in the patients.

Preliminary work is already done by the Investigator in treatment/cure of HIV/AIDS in the year 1991-1993- some HIV/AIDS cases referred by King George Hospital at Vizag. And Kakinada General Hospital-Andhra Pradesh(lndia).

### BRIEF SUMMARY OF THE INVENTION

HIV/AIDS, which is global problem is the syndrome associated with depletion of T4 lymphocytes and profound cellular immune deficiency.

According to Ayurveda AIDS is due to disturbed normalcy of normal immune system. It is a venture to put before the medical profession the drugs that can arrest and cure the diseases HIV/AIDS and Cancer formulated by the inventor.

The drugs Avalmic—I & II, which when taken into consideration the total internal environment of the body (the whole system) and the local condition concurrently, they boost the vitality (immunity) of the individual. The drugs promote and maintain the immune mechanism at the optimum level by raising the body's own defense mechanism. This is also an ideal therapy in all immunological disorders including dreaded diseases-HIV/AIDS, Cancer etc.

By using these 4 combinations of Ayurvedic drugs difference in CD4 absolute count came back to normal with in 4 to 6 months after the commencement of treatment. Gradually viral load comes down to absolute NEGATIVE within 1 to 3 years i.e. if the viral load per each ml of plasma numbered in hundreds in a patient; With the use of medicine it will turn into HIV negative in one year; On the other-hand if it is in thousands in 2 years, in ¼ to 1 million—in 3 years. But some variations or difference we have seen when patient does not follow precautions with regard to sexual intercourse and other restrictions. All symptoms disappear completely within 2 to 3 months. In case of Western Blot also we have observed all one after the other the bands will come to negative/disappear within 1 to 3 years depending upon the viral present in the body.

To get complete response in very advanced cases KAPH YOG & MEDHAMRUT are also given for 2 to 4 months following the usage of AVALMIC-I & AVALMIC-II according to the condition. Cases that were declared positive by ELISA, WESTERN BLOT AND VIRAL LOAD tests, where CD4 absolute counts decreased. All these cases were proved Negative by all the Diagnostic methods done by various other Standard Diagnostic Laboratories in the Country.

### BRIEF DESCRIPTION OF THE SEVERAL VIEWS OF THE DRAWING

FIG. 1: The accompanying figure is the statistical graph of a patient treated by the inventor, his age is 35 years, Male person and diagnosed as HIV patient with CD 4 count of 331 before treatment and 1672 after treatment which shows a increase in the CD 4 count after treatment. And also Viral load of 37889 before treatment and <20 after treatment, which shows normal in the viral load after treatment by the inventor.

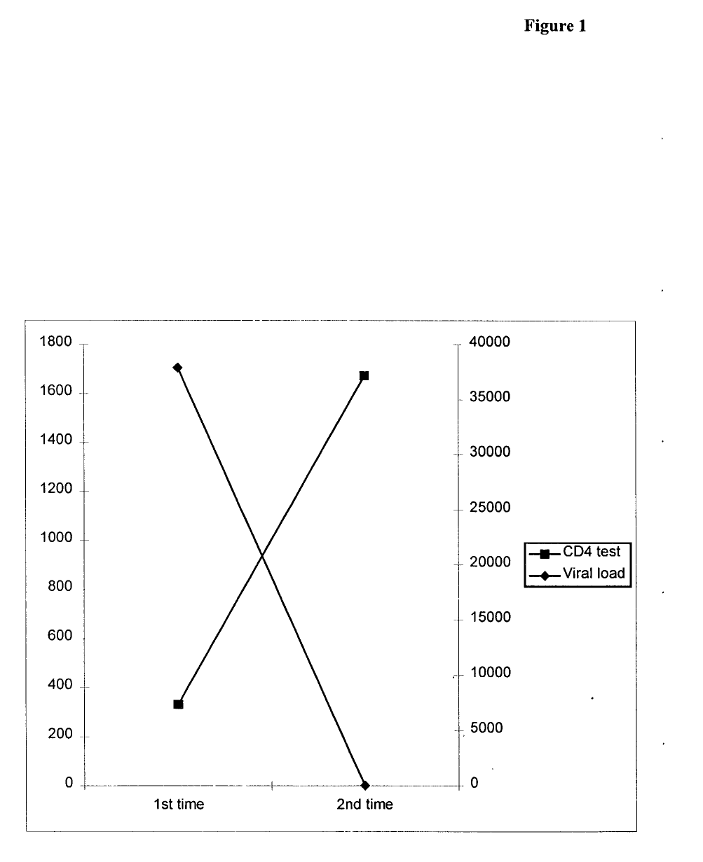

Patient: Male. Age-35years

| Diagnosis | 1st time | 2nd time | Difference |
|---|---|---|---|
| CD4 test | 331 | 1672 | 1341 |
| Viral load | 37889 | 20 | 37869 |

The above table shows increase trend in Absolute CD4 count from 331 to 1672 and descendence in Viral Load I.e. from 37889 to <20.

FIG. 2: The accompanying figure is the statistical graph of a patient treated by the inventor, his age is 56 years, Male person and diagnosed as HIV patient with CD4 count of 850 before treatment and 1568 after treatment which shows a increase in the CD 4 count after treatment. And also Viral load of 6497 before treatment and 2245 after treatment which shows reduction in the viral load after treatment by the inventor.

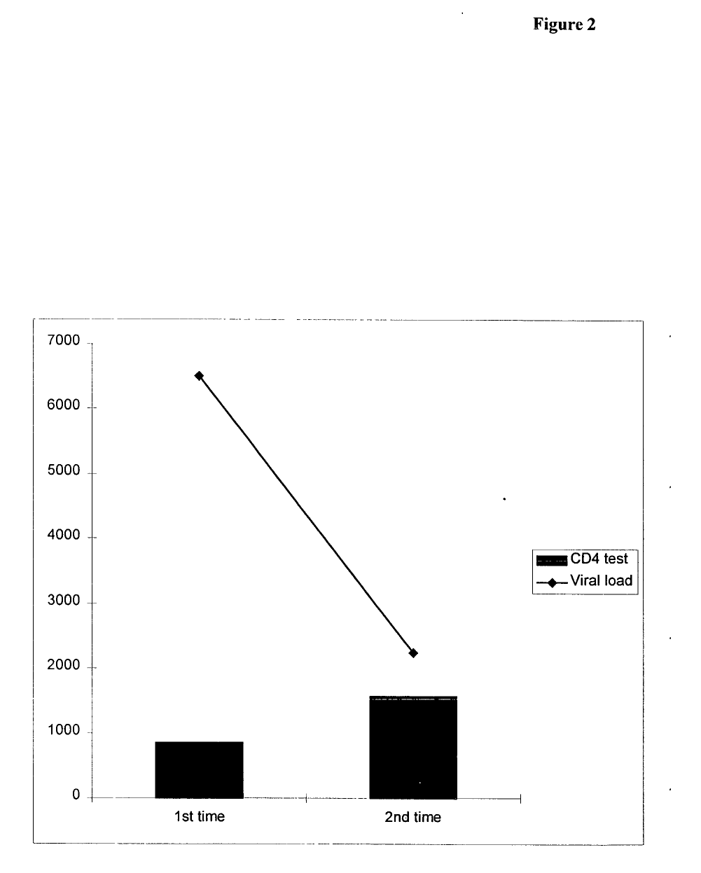

Patient: Male, Age: 56 Years

| Diagnosis | 1st time | 2nd time | Difference |
|---|---|---|---|
| CD4 test | 50 | 1568 | 718 |
| Viral load | 6497 | 2245 | 4252 |

The above table shows increase trend in Absolute CD4 count from 850 to 1568 and descendence in Viral Load I.e. from 6497 to 2245.

FIG. 3: The accompanying figure is the statistical graph of a patient treated by the inventor, his age is 26 years, Female person and diagnosed as HIV patient with CD4 count of 474 before treatment and 1003 after treatment which shows a increase in the CD4 count after treatment. And also Viral load of 18723 before treatment and 3895 after treatment which shows reduction in the viral load after treatment by the inventor.

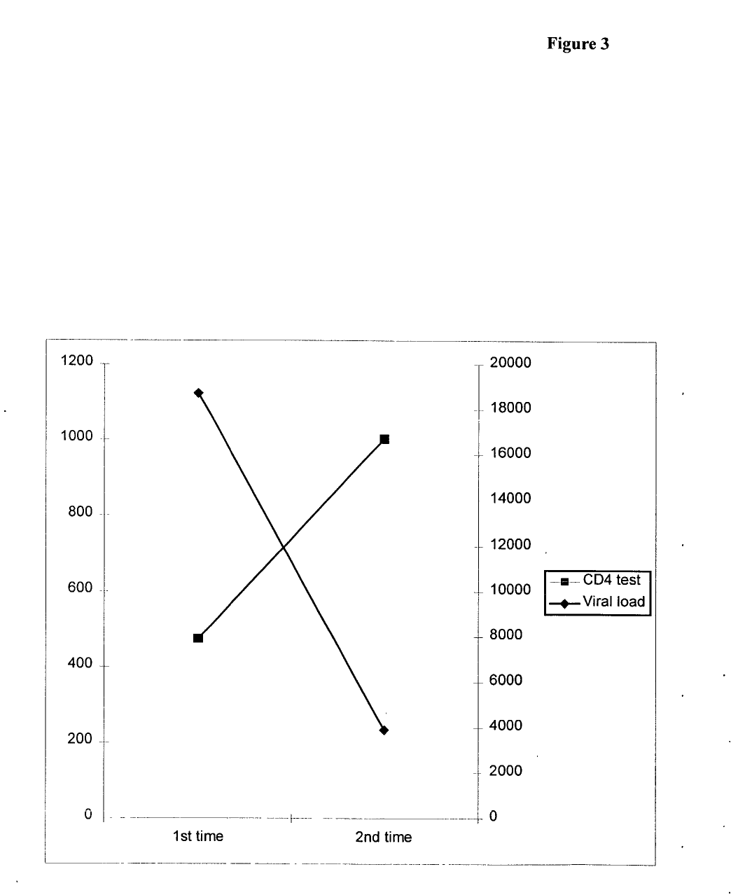

Patient: Female Age 26 years

| Diagnosis | 1st time | 2nd time | Difference |
|---|---|---|---|
| CD4 test | 474 | 1003 | 529 |
| Viral load | 18723 | 3895 | 14828 |

The above table shows increase trend in Absolute CD4 count from 474 to 1003 and descendence in Viral Load I.e. from 18723 to 3895.

### DETAILED DESCRIPTION OF THE INVENTION

The therapy presented here for raising/maintaining CD4 count and reducing Viral load and to get the results negative for AIDS patients makes use of the combinations of botanical plants to prepare so called Ayurvedic drugs.

In Ayurvedic a unique clinical entity was described about 3,000 years back in the Samhita period which is identical in the etiological, clinical features and prognosis of dreaded disease HIV/AIDS. Sushruta Charya used the term “VYAVAYA SOSHA” while narrating the evil effects (Symptoms) of perverted Sex indulgence. The same term is also used while recommending the line of treatment.

It is interesting to note ‘SOSHA’ (name of the disease syndrome) as one of the communicable disease i.e. spreading from one to another person (Samkramita diseases described in the Ayurvedic classics).

The following are the four formulations, which are concurrently given.

1. AVALMIC-I.
2. AVALMIC-II
3. KAPHAYOGA
4. MEDHAMRUTHA

The following are some of the symptoms which may be seen in HIV/AIDS patients according to guidelines of Indian Council of Medical Research (ICMR)-Ministry of Health and Family Welfare—Govt. of India—(New Delhi):—

Feverishness/Body Heat, Loss of Appetite, Anorexia, Indigestion, Nausea, Vomiting, Abdominal Pain, Weight Loss, Diarrhoea, Dysentery, Thirst, Body Pains, Joint Pains, Back-ache, Cramps, Forgetfulness, Oedema, Throat Pain, Angular Stomatitis, Glossitis, Difficulty in Swallowing, Difficulty in Breathing, Bleeding Gums, Skin Disorders, Lumps, Fatigue, Stranguria, Night Sweats, Cough, Breathlessness, Head-ache, Swelling, Yellowish Discoloration, Constipation, Ulcer.

Within 24 to 48 hours body heat (feverishness) subsides completely and the end of 1st week symptoms like nausea, vomiting, loose motions, etc. subsides and regains appetite. By the end of 2nd week headache, anxiety, neuralgia etc., subsides, except some skin and other problems, if the patient is not having any associated diseases like Cancer, T.B., Malaria, Typhoid etc. By the end of 3rd week there will be weight gain. By the end of 2nd & 3rd month all the symptoms completely subside. Gradually viral load comes down to negative within 1 to 3 years depending upon the viral quantity present in the body and almost all cases absolute CD4 comes to normal during or after 4 months completion of this treatment.

Therefore all organs come to normal as before. But with the use of any other system of medicine i.e., Allopathic, Homeopathic, or even other Ayurveda, if the patient spends much of money, the body heat/feverishness does not subside till his death (unless he uses these medicine). It means drug action starts to destroy/reduction of virus within minutes and to normalize the organs to function.

The most advantage of this medicine (therapy) is one can use Allopathic medicines—Simultaneously in the management of acute complications without any side effects as it is not contra-indicated with any Allopathic medicine.

The inventor is giving treatment for 4 months only but it takes 1 to 3 years to get negative, depending upon the viral present in the body. Also when the patient follows precautions and other restrictions till he gets negative, provided he is not attacked with associated diseases like T.B., Cancer, etc. (before, during & after) treatment. In such cases it is not possible to get results with 4 months treatment.

The inventor has sent the drugs for vitro testing at the highest laboratory in the country and found encouraging results. The four drugs used in the treatment/cure of HIV/AIDS were tested separately and found:

The toxicity of the drug was tested, in vitro, on the cell line that is used in the assays for Anti-HIV activity. The highest concentration of the drug that did not have any toxic activity on the cells in the culture was tested for anti-HIV activity: Anti-HIV activities were tested against the cell free as well as cell associated (intracellular) virus.

When tested against cell free virus, there was reduction in the virus growth.

COMPOSITION:

COMPOSITION—I: {Avalmic-I}: Pulaparni, Vallijam, Palandu, Karanajaka, Chanaka, Sigda, Bharbura and Herbal powder base.

DOSE:

AVALMIC-I-100 to 300 mg:

Adult: 1 cap twice a day for 2 months after break fast and dinner.

Children: 100 to 200 mg (½ to one cap) once or twice a day age×3 days (i.e. if age is 3 years=3×3=9 days.)

This unique combination of natural ingredients boosts the natural immunity of the human body. It promotes and maintains the immune mechanism at optimum level by raising the defense mechanism. And destroys the causative factor HIV Virus and some other infections.

It normalizes the cellular activity anywhere in the body and it is the drug of choice in early stage of carcinoma, sarcoma, leukemia, lymphoma and AIDS. So it cures human disorders like loss of weight, cocexia and activates cerebral and neural activity.

It acts as broad spectrum anti-biotic. Within 2 months all organs function normal as before and Absolute CD4 raises and viral growth reduces according to the virus present in the body in combination of all given compositions.

The Patients are instructed to follow strict dietary regimen to facilitate quick recovery from the disease.

CONTRA-INDICATIONS & SIDE EFFECTS: No known contra-indications & side effects.

Herbal plants procured in their natural form and cleaned to remove dust, rotten part and any other foreign matter. These parts of the plants are dried separately to remove excess of moisture and reduced to Bhasmam (Ash) by the process called “PUTAM”. Thus the natural ingredients converted to Herbal Bhasmam (Ash) as per preparation percentage by Weight.

Seven different plant parts are mixed in fixed amount. The process of preparation cleaning, drying, mixing etc finally administered into dried herbal powder and later filled into gelatin capsule. Total weight of capsule—100 mg to 300 mg

COMPOSITION-II: {Avalmic-II}: RASNA, PARADA, PUSHAPAKASEES, SULVA, AMLAKI and HERBAL POWDER BASE—Quantity Sufficient.

DOSE:

AVALMIC-1′-150 to 350 mg:

Adults: 1 cap once a day up to 4 months after lunch.

CHILDREN: 50 TO 100 MG (¼ TO ½ CAP) ONCE OR ALTERNATE DAYS UPTO 1-2 MONTHS ACCORDING TO THE AGE AND SEVERITY OF THE DISEASE.

This combination of drug is indicated for repair of the structure by destroying the virus and to promote physiological activity of the tissues. It also purifies and restores the blood.

It has an established action as Rakthaposhaka. It improves the production of Erythrocytes and improves the total count of R.B.C. and acts on the infective condition caused by non-specific and specific organisms.

It acts as broad spectrum anti-biotic to bring all organs to function normal as before and Absolute CD4 becomes normal and viral growth reduces in combination of all given compositions.

After treatment almost all patients' absolute CD4 count Increased/became normal, if they are not having any associated diseases especially like T.B, Cancer etc.

Therapeutically this unique drug develops vital power (ojas) of the blood. It further repairs various tissues and promotes this physiological activity of the cells.

CONTRA-INDICATIONS & SIDE EFFECTS: No known contra- indications & side effects.

Three types of plant parts are mixed into later triturated in a mortar until a uniform mixture formed and then triturated with pure another three types of ingredients, mixed with uniform mixture powder with the base until very thin uniform mixture (powder) is formed.

The mixture of ingredients with required doses is then filled into Gelatin Capsule of appropriate size (i.e. 150 mg to 350 mg).

COMPOSITION—III: Kaphyog: Amalaki, Mahanimba, Tilapushpa, Vajravalli, Ankola, Tulasi, Gandhamula, Amruta Trikatuka.

DOSE:

KAPHYOG -100 TO 300MG:

Adults: 1 cap twice a day for 4 months after breakfast and dinner.

Children: 50 to 100 mg (to ½ cap) after breakfast or dinner up to 1-2 months.

This composition rectifies (reforms) gradually all mucus membranes in the body by reducing the virus.

After treatment almost all patients' absolute CD4 count Increased/became normal and reduction in viral growth in combination of all given compositions; if the patient not having any associated diseases especially like T.B, Cancer etc.

CONTRA-INDICATIONS & SIDE EFFECTS: No known contra-indications & side effects.

All nine different plant parts mixed in fixed amount in the process of preparation cleaning, drying, mixing etc herbal contents (ingredients) are powdered and sieved, finally mixed together until uniform mixture (powder) is formed, finally administered into dried herbal powder filled into gelatin capsule. Total weight of capsule 100 mg to 300 mg

The mixture of ingredients with required doses is then filled into Gelatin Capsule of appropriate size (i.e. 100 mg to 300 mg). This Herbal Drug Capsule is ready to use, easily acceptable to the patients and normalizes the cellular activity anywhere in the body.

COMPOSITION—IV: Medhamrut: Ananta, Varahakarni, Silaja, Silajit, Talpotali, Mahanimba, Madhuyasti, Chakrangi, Ratanpursha.

DOSE:

MEDHAMRUT -100 to 300 mg: After completion of AVALMIC-I

Adults: 1 cap twice a day for 2 months after breakfast and dinner

Children: 50 to 100 mg (¼ to ½ cap) after breakfast or dinner upto 15 days to one month.

This composition tones up neural activity. It enhances natural sleep; relieves insomnia, increases memory and enhances intellectual capacity.

After treatment almost all patients' absolute CD4 count Increased/became normal and reduction in viral growth in combination of all given compositions, if the patient not having any associated diseases especially like T.B., cancer etc.

CONTRA-INDICATIONS & SIDE EFFECTS: No known contra- indications & side effects.

All nine different plant parts are mixed in fixed amount in the process of preparation cleaning drying, mixing etc herbal contents (ingredients) are powdered and sieved. Finally mixed together until uniform mixture (powder) is formed, finally mixed with pure other two ingredients triturated until uniform mixture powder is formed and administered into dried herbal powder filled into gelatin capsule. Total weight of capsule-100 mg to 300 mg.

The mixture of ingredients with required doses is then filled into Gelatin Capsule of appropriate size (i.e. 100 mg to 300 mg). This Herbal Drug Capsule is ready to use, easily acceptable to stimulate the peripheral and central nervous system.

This four herbal composition tested in vitro/vivo, even highest concentration of the drug has no toxic & side effects.

“Anubootha Yoga” system were giving encouraging results in ancient days but some of the Classics or traditional practitioners concealed this traditional formulations, not bringing them into light. The modern medicines in the primary health care have many side effects and so once again the traditional formulations have been revived, as there are no side effects. So, Continuing efforts to document these formulations are necessary to preserve this information.

This combination therapy acts by inhibiting opportunistic infections with concurrent stimulation of the immune system. By increasing the body's ability to fight against various infectious microorganisms, the symptoms associated with AIDS are subsided within 2 months except Skin/Dermatological Disorders. Weight increases up to 2 to 4 Kgs within 4 months. After completion of theraphy, in some cases weight may sometimes increase up to 8 to 10 Kgs. Virtually, ‘good health’ is maintained in all Patients using the present combination of the present invention shown by the increase in body weight, or less than 5% decrease from the starting body weight (if patient is having Oedema).

Almost all Patients come to our institution after confirmation of their disease from various laboratories and referred by concerned Doctors/Institution after using some medicine for months to years and some other patients come after loss of their spouse (wife/husband).

Patients can stay at home and use medicines without any side effects. They need not stay as in-patients. Now a days, Allopathic medicines are costly, much side effects/non reduction of virus and not possible to regain the health in all aspects.

All age group people can use these medicines. They work as broad spectrum anti-biotic to the human body. Medicine is taken in less quantity but shows high/Quick result. Health improves a lot.

FEATURES: By using these drugs (Avalmic—I, Avalmic—II, Kaphyog & Medhamrut) formulated by inventor Manufactured at M/s G.P.Pharmaceuticals (Ayurvedic Research Medicine Division) treated at G.P.Institute/Hospital of Alternate Therapies and Research Center. Absolute CD4 & CD8 count became normal within during/after commencement of treatment. Gradually viral load comes down to absolute NEGATIVE within 1 to 3 years i.e. if the viral load per each ml of plasma is numbered in 100's in a patient, with the use of medicine it will turn into HIV negative in 1 year. On the other-hand if it is in 1000's in 2 years, in 1,00,000's in 3 years. But symptoms disappear completely within 2 to 3 months. (But the treatment is only 4 months for any patient) when the patient follows precautions and other restrictions till he gets negative, provided he is not suffering with any associated disease like T. B., Cancer, etc. (before, during & after) treatment. In such cases it is not possible to get results with 4 months treatment.

Certain patients are tested under Western Blot, as Viral Load Test is not affordable by all. In such cases—If all bands disappear within 1 year, then it is ascertained that the patient must be having hundreds of virus in 1 ml of plasma. When all bands disappear within 2 years, it is concluded that the patient must be having thousands of virus in 1 ml of plasma i.e. gradually all the bands disappear within a period of 2 years. Similarly if bands disappear within 3 years it is understood that the patient must be having ¼ to 1 million of virus in 1 ml of plasma i.e., gradually all bands will disappear within a period of 3 years.

It is practical experience with recorded evidence (diagnostic reports) that HIV/AIDS patients who are under-going excess of sexual intercourse and do not. follow other restrictions after completion of treatment, results in decrease of CD4 count and again raising of viral load in the body. Hence it is advisable that after completion of 4 months course—follow monthly once or 15 days once with the usage of condoms until negative occurs.

We declare 99% cure, if the patient is not having any dreaded disease like T. B., Cancer etc. without any side effects. In case of patients with associated diseases like T.B. etc. it is curable up to 75 to 85% depending upon the severity of the T.B. etc (Provided he uses allopathic medicine for T.B. along with this medicine combined).

Category of patients shall be based on past history of illness, pregnant and lactating women, children, adults, and old people. The inventor has proved treatment/cure in Allopathy exclusion criteria also with treated HIV/AIDS patients. Successfully without any side effects. i.e. Past history of convulsions or cardiac diseases and currently requiring medication for control, those having taken AZT in the immediately preceding 4 weeks, Pregnant and lactating women. For patients with associated diseases—T.B., Dermatological Disorders, Cancer & Psychiatric Disorders the Inventor while continuing his treatment has referred them to T.B. & Chest Specialist, Dermatologist, Oncologist and Neuro-Psychiatrist etc where they found remarkable improvement in clinical, radiological and bacteriological aspects of their patients without any side effects.

Treatment: Patients will be randomly allocated in equal proportion to the traditional remedy (Placebo) after stratification by stage and presence/absence of any dreaded diseases (i.e. T. B. Typhoid, Malaria). The duration of therapy is 4 months at G.P.lnstitute /Hospital of Alternate Therapies and Research Center.

Investigations during treatment: appropriate investigations will be undertaken at periodic intervals (Stage II, III, and IV).

Routine Examinations and Assessments: Before commencement of the trial/treatment. General information will be elicited, complete medical history, physical examination, HIV Screening (ELISA), HIV Confirmation (Western Blot) tests, viral load (HIV-RNA-PCR- ULTRA-QUANT). CD4 & CD8 cell counts will be undertaken if the patient can afford.

General Management: Consent will be obtained from every patient, strict confidentiality of all data collected will be maintained to prevent embarrassment to HIV patients, as HIV infection/AIDS carries a social stigma.

All patients will be managed on out patient basis (Stage II, III & IV). Each patient will be issued the appropriate drug in systematic manner. Patient/his relative/friend will be instructed to know the condition of the patient after 15 days (after 1st dose). And a record will be maintained of the drugs consumed. Periodic assessments, if any, made by any other physician who has medicated the patient is also maintained.

Patients will be instructed to avoid all other forms of medication during this treatment for HIV infection, any other associated illness, they should report to the physician immediately and take appropriate treatment (instructed to take allopathic medicine).

Laboratory Methods: Serology kits ELISA, Western blot, viral load test. There are various blood tests that can help to show the strength of immune system or how much virus is present in patients' body. In the past most people with HIV infection learned about the T-cells tests (CD4 cells). But in recent years the “Viral Load” test has become very important as well. These tests can help plan of treatment. The viral load is the amount of HIV measurable in the plasma. The test is a relatively new tool for determining how fast HIV is reproducing in the body.

People with recent infection often have viral loads of a % to 1 million/more particles in a milliliter of plasma. After 6 months following initial infection most people will have antibodies to HIV in their plasma and their immune system will have reduced the viral load in the plasma to some lower level. Studies have shown that people with the highest viral levels after 6 months are those whose immune system cells are most rapidly destroyed; people with the lowest viral levels have the lowest progression of HIV infection. Even high viral load test results do not require aggressive treatment for HIV.

Absolute CD4 count test: During and after completion of the therapy almost all Patients absolute CD4 Count increased or become normal if they are not having associated diseases especially like T.B.etc.

Few example cases proved by Absolute CD4 count as follows.

Absolute CD4 count of some of the treated HIV/AIDS Patients

| | | Before/During | |
|---|---|---|---|
| Patient age, sex | Schedule of therapy | treatment | After treatment |
| 1. 40, Male | 4 months | 213 | 632 |
| 2. 20, Female | 4 months | 145 | 328 |
| 3. 27, Male | 4 months | 164 | 423 |
| 4. 30, Male | 4 months | 266 | 355 |
| 5. 32, Male | 4 months | 126 | 620 |
| 6. 25, Male | 4 months | 171 | 761 |
| 7. 26, Male | 4 months | 191 | 324 |
| 8. 28, Male | 4 months | 232 | 983 |
| 9. 56, Male | 4 months | 850 | 1568 |
| 10. 26, Female | 4 months | 474 | 1003 |
| 11. 35, Male | 4 months | 331 | 1672 |

Elisa test:: Through Elisa Test, one can only know whether HIV is present or not but cannot know how much virus is present. Even after the treatment, also he may get the word positive only through this test but not the exact position. If the patient had 10% or 90% of virus left in his body, the result would be shown positive. In other words, what percentage of virus is present in the patient's body will not be known. Patients, who are eager to know their result, lose confidence, as even if the percentage of virus is getting reduced, it cannot be brought to light through this test. That is each time the patient is tested, even if he is having 10% or 90 % of HIV virus also his report shows positive. Until the patient gets negative i.e. in some cases it may taken even 1 to 3 years. Meanwhile any associated disease/fever may develop, then at that time, it is general thinking that it is due to HIV. So some persons belonging to the medical line also think in the same way and give suggestions to patients that it is due to HIV/AIDS but it is due to T.B. or any other associated disease fever i.e. Typhoid or M.P. etc. HIV/AIDS patients usually have fever or he suffers with feverishness/body heat. If he is having fever he must be having T.B, Typhoid or M.P.

During/After completion of therapy turned HIV Positive to HIV Negative within short time through Elisa test. Some example Cases as follows.

Case 1: 21 years, Male (HIV WITH BRAIN TUMOUR)

Duration of treatment: Dt.9.5.2000 to 8.9.2000 (4 months treatment)

Before treatment: This patient suffered from Brain Tumor at the age of 5 years. He was operated for the same at one of the government institute. After that he was in better health till 15 years. Later he was attacked with fits and was treated at one government institute. Later he has undergone C.T. Scan and blood test at one government institute on Dt.2.5.2000. The outcome was that he was suffering with Brain Tumor. The Neurologist at one government institute informed that Tumor was growing and he cannot be operated as he was infected with HIV/AIDS. Then he approached our Institute. Before treatment he suffered with symptoms like Feverishness/Body Heat, Headache, loss of appetite, Fits etc and 18 other symptoms.

During treatment: After 15 days treatment the symptoms like Body heat/Feverishness, anorexia, cough subsided. In about 3 months time other symptoms were completely subsided.

After treatment: After 4 months treatment with all diet restrictions as on 9.9.2000 he was tested at one pathology Center done Elisa test- report showed HIV—I & II antibodies negative. Again on 18.9.2000 at one diagnostics Center he has undergone Elisa test report showed for HIV Negative. Later he has undergone operation for Brain tumor and the outcome was great success.

OUR OBSERVATION: This patient is having viral load in hundreds per ml plasma, so HIV-I & II infection turn to Negative with in 1 years. Same as above in Western Blot test HIV-I & II all bands turned Negative. It means the patient may be having Viral Load in 100's per 1 ml plasma.

Case 2:—AGE: 1 year—06 months Female Baby(HIV INFECTED INFANTS)

Got infected with HIV I through her HIV infected mother.

Duration of treatment: 10.1.1999 to 09.02.1999.

Before treatment: She delivered the female baby on 11.10.99. Father Aged 25 male also HIV-I- POSITIVE. On 11.10.99 the baby was tested for Elisa—HIV infection and found “positive”.

All the three members including the Baby have taken our medicines for 1 month as per our dosage schedule and found relief very satisfactorily from all symptoms like Fever, Diarrhea etc.

After treatment: The baby acquired HIV-ve (negative) result exactly after one-year i.e. on 3-1-2000. It is evident that HIV infected children are amenable to our treatment.

OUR OBSERVATION: This patient is having viral load in hundreds per ml plasma, so HIV—I & II infection turned to Negative with in 1 year. Same as above in Western Blot test HIV—I & II all bands turned Negative. It means the patient may be having Viral Load in 100's per 1 ml plasma.

Few Example cases report shows after completion of therapy through Elisa test HIV—Positive cases turned to HIV-Negative.

| PATIENT | BEFORE | AFTER | |
|---|---|---|---|
| AGE, SEX | TREATMENT | TREATMENT | PROGNOSIS |
| 1. 1½, Female | ELISA TEST | ELISA TEST | After 1 year |
| | HIV-1 - Positive | HIV-1 - | HIV - Negative |
| | | Negative | |
| 2. 20, Female | ELISA TEST | ELISA TEST | During 4^{th} month |
| | HIV-1 - Positive | HIV-1 - | HIV - Negative |
| | | Negative | |
| 3. 32, Male | Elisa test | ELISA TEST | During 2^{nd} month |
| | HIV-1 - Positive | HIV-1 - | HIV - Negative |
| | | Negative | |
| 4. 18, Female | W. B. test | ELISA TEST | During 4^{th} month |
| | HIV-I - Positive | HIV-1 - | HIV - Negative |
| | | Negative | |
| 5. 21, Male | Elisa test | ELISA TEST | After 1^{st} month |
| | HIV-I - Positive | HIV-1 - | HIV - Negative. |
| | | Negative | |
| 6. 54, Male | Elisa test | ELISA TEST | During 4^{th} month |
| | HIV-I - Positive | HIV-1 - | HIV - Negative |
| | | Negative | |

Western Blot Method: In case of Western Blot Test the patient can only know how many bands are present and after treatment how many bands are reduced. Therefore, this test is useful than Elisa test as it shows how many bands disappeared in certain period. But at the same-time it is not possible to know in how many days/months negative occurs till disappearance of all bands. In case of viral load test it has become very easy for us as well as to the patients to know, because, when they are tested through this test they get confidence as to in how many days they can get negative and be assured of it - when he follows these precautions.

During/After completion of therapy turned HIV Positive to HIV Negative within short time through Western Blot tests. Some example Cases as follows.

Case 1: 21, Male—70 Kgs—Duration of treatment: 13.7.2000 to 14.11.2000

Before treatment: Before approaching to the Research institute He was tested in Elisa and found HIV Positive.

Symptoms: He suffered with symptoms like Fever, Fatigue, Sweats, Loss of appetite, Body pains, Pains of limbs, Joint pains, Thirst, Sleeplessness, Fear, Abdominal pain.

During treatment: After 15 days of treatment Fever, Fatigue, Sweats, Loss of appetite subsided and after one to three months symptoms like Body pains, Pains of limbs, Joint pains, Thirst, Sleeplessness, Fear, Abdominal pain also subsided.

After treatment: After treatment of 6 months, he was tested CD4 & CD8 853/739 at one standard lab, on 6.4.2001 and in Western blot test of HIV - found all bands negative and only serum control band positive. His weight increased from 70 kg to 74 kg. Now the patient is healthy and able to do his own profession—business.

Our Observation: This patient is having viral load in hundreds per ml plasma, so HIV-I & II infection turn to Negative with in 1 years. Same as above in Western Blot test HIV-II & I all bands turned Negative. It means the patient may be having Viral Load in 100's per 1 ml plasma.

Case2: 40,Male—Wt: 70 Kgs. Merchant, married, period of treatment: 28.3.1997 to 27.7.1997.

The patient before approaching our institute has undergone western blot test on 14.3.97 for HIV-I & II—Positive.

The Patient before approaching our institute on

8.4.97-CD4 & CD8-213-505

1.8.98-CD4 & CD8-355-223

18.4.98-CD4 & CD8-521-1135

13.2.99-CD4 & CD8-632-1142(In these tests CD4 increased on 8.4.97-213 to on 17.2.99-632.)

On 29.10.99-western blot test showed Negative HIV-II-Negative and HIV-1-8 bands Negative and 2 Bands positive i.e. P-51 & P-17.

His wife was aborted twice before treatment. Now she is blessed with a Son and both are healthy. All three persons reports showed Negative after 3 years.

Our Observation: This patient is having Viral Load in ¼ to 1 million per ml of plasma, so HIV-I & II infection turned negative within 3 years. Same as above in Western Blot test HIV-I & II all bands turned Negative. Few Example cases report shows after completion of our treatment W.B. Method Bands disappeared within 1 to 3 years.

| PATIENT | BEFORE | AFTER | |
|---|---|---|---|
| AGE, SEX | TREATMENT | TREATMENT | PROGNOSIS |
| 1. 40, Male | W. B. Test - | W. B. Test | Within 3 Years |
| | HIV-1 - 10 | HIV-1 - 8 | HIV - Negative |
| | Bands | Bands | |
| 2. 44, Female | W. B. Test - | W. B. Test | After 5 months HIV |
| | HIV-1 All Bands | HIV-I all | Negative |
| | Positive | Bands | |
| | | Negative | |
| 3. 24, Female | Elisa test | W. B. test | Within 3 years HIV |
| | HIV-1 - Positive | HIV-1-7-bands | Negative |
| | | Negative | |
| 4. 18, Female | Elisa test | W. B. test | Within 3 years HIV |
| | HIV-I - Positive | HIV-1-9-bands | Negative |
| | | Negative | |
| 5. 25, Male | Elisa test | W. B. test | Within 3 years HIV |
| | HIV-I - Positive | HIV-1-9-bands | Negative |
| | | Negative | |
| 6. 21, Male | Elisa test | W. B. test | Within 1 years HIV |
| | HIV-I - Positive | HIV-1-9-bands | Negative |
| | | Negative | |

Viral load/HIV-RNA-PCR-ULTRA QUANT: Through Viral Load Test a patient can know his exact condition i.e. if the patient is having hundreds of virus in 1 ml of plasma, then he will get negative within 6 months to 1 year. On the other hand, if the patient is having thousands of viruses in 1 ml of plasma, then he will get negative within 1 to 2 years. In addition, if the patient is having ¼ to 1 million of virus in 1 ml of plasma, then he will get negative within 2 to 3 years.

During/After completion of therapy turned HIV Positive to HIV Negative within short time through HIV-RNA-PCR-ULTRAQUANT (Viral Load) test reports, which have shown astonishing results i.e. HIV Virus destroyed per ml of Plasma.

Some example Cases as follows.

### EXAMPLE 1

### Age: 35 years, Male

Duration of treatment: 21.7.2001 to 22.11.2001 (4 months)

Symptoms: Body Heat/Feverishness, Joint Pains, Loss of Appetite, Heaviness in the Stomach.

Before treatment: This patient contacted local Doctor for Fever and general weakness. At that time the Doctor advised him to undergo certain tests. Then he was tested at Central Lab self reference, on 10.2.2001—for HIV I & II showed HIV I Reactive & HIV—II Non Reactive. He used some retrovirus medicines and was tested on 24.2.2001 he was tested at one standard lab for CD4 & CD8 count—535/1147. Again he was tested at one standard lab on 25.6.2001 for CD4 & CD8 count—421/1004. Here though he used the above said drugs his CD4 count reduced to 535 to 421.

During treatment: Later after 15 days treatment at our institute he got relief from Body heat/feverishness. After taking treatment for 1 month he was tested at one of the Standard Lab.As on 31.7.2001, his HIV-RNA-PCR- ULTRA QUANT (Viral load) 37889 and his CD4 came to normal i.e.331.

After completion of treatment on 25.9.2001, his HIV-RNA-PCR-ULTRA QUANT(Viral load)<20 and CD4-1672. Here his CD4 count raised from 331 (during 15 days treatment) to 1672(after completion of treatment) and viral load shown in range.

It shows that using these herbal medicines his CD4 count raised remarkably and viral load shown in range astonishingly without any side effects.

The difference in viral load for this patient—

| Viral Load | Viral Load | Viral | |
|---|---|---|---|
| Before 1 month | During 2 month | Destroyed | Viral |
| Treatment | Treatment | Within 2 months | After 4 months |
| 37889 | 8600 | 29289 | <20 (In range) |

### EXAMPLE 2

### 63 years, Male (HIV Infected with Cardiology) Duration of Treatment: 19.1.2001 to 8.5.2001. (4 months)

Symptoms: He suffered with Body Heat/feverishness, Swelling, Breathe- less-ness, Loss of Appetite, Heaviness in the Stomach, Cough, Oedema (Both Limbs), Not able to walk for more distance and 14 other symptoms.

Before Treatment: This patient is suffering with heart problem. He is using pacemaker since 1992. He is getting treated at one government institute. He has approached them on 14.12.2000 again, as he wanted to change the pacemaker. At that time, he was tested for HIV referred by cardiologist. The out-come was that—HIV RNA PCR Ultra Quant (Viral load) Report—Showed 89126 (1 ml of blood).

During Treatment: Later when this patient approached us. After 15 days of treatment he got relief from symptoms like Breathe-less-ness, Body Heat/Feverishness, Swelling, Oedema, etc and appetite improved. After 2 months treatment all other symptoms subsided.

After Treatment: He started treatment on 19.1.2001. After 4 months treatment i.e. as on 3.7.2001, his Viral Load showed remarkable change i.e. it has come down to 14555 and his CD4 came to normal i.e. 608 as on 3.7.2001. Here one must note that in 1 ml of plasma patient is having virus of 89126 and after treatment reduced to 14555. So 89126-14555=75571 virus is destroyed in 1 ml of plasma, that means in whole body virus may be destroyed in millions.

The above viral count result is only after completion of 1½ month of treatment and hence as day-by-day viral gets destroyed and immunity increases. As a result the patient will get negative result in few months. All this depends upon the immunity of the body and behavior of the patient and follow-up of our restrictions.

Here we are presenting some case reports. After the treatment HIV Positive cases viral load shown remarkable change.

### EXAMPLE 3

### Male, 27 yrs., Duration of Treatment: 30.5.2000 to .10.2000 (4 months)

Before Treatment: Before approaching the inventor, this patient was suffering with symptoms like Body heat/Feverishness, Palpitation, Forgetfulness, Fear, Falling of Hair, Indigestion, Thirst, Headache, Night Sweats, Abdominal Pain, and Herpes. As on 13.3.2000, this patient was tested at one diagnostics Center for Western Blot Test and confirmed with HIV-I infection all bands positive HIV-II Negative.

During Treatment: After using this medicine for 15 days his appetite improved and other symptoms like Body heat/Feverishness, Thirst, Night Sweats subsided. After 2 months other symptoms like Palpitation, Forgetfulness, Fear, subsided. His weight increased by one-kg i.e. from 65 Kg to 66 Kg.

After Treatment: After completion of 4 months treatment as on 10.10.2000 his HIV RNA PCR—ULTRA QUANT report showed 31018 and on 26.6.2001 his Viral Load showed 22759. i.e. in 1 ml of plasma, 31018-22759=8259 virus is destroyed. His CD4 came to normal i.e. 677. Patient's weight also increased by 2 kg i.e. from 65 Kg to 67 Kg as on 29.9.2000.

The difference in viral load for this patient—

| Viral Load per 1 ml of | | |
|---|---|---|
| plasma After | Viral Load 1 ml of plasma | viral Destroyed |
| 4 months Treatment | 11 months After Treatment | within 7 months |
| 31,018 | 22,759 | 8,259 |

### EXAMPLE 4

### Male, 56 years;

Duration of treatment: 28.6.2001 to 29.10.2001 (4 months);

Symptoms: Fatigue, Body Heat/feverishness, Virtigo, Dullness, irritability, Diabetes, B.P., Light Heart Pain, Palpitation, Loss of Appetite.

Before treatment: The patient before approaching the Inventor was suffering with heart problem. He was taking treatment at one government institute. At that time the doctor referred for Angiogram and blood test for HIV. In Western Blot Test the result was HIV-I all bands positive and HIV-II negative. Before treatment he was tested at one standard lab on 28.6.2001 HIV-RNA-PCR-ULTRA QUANT (Viral load)-6497. After 2 months treatment i.e. as on 29.9.2001, his Viral Load showed remarkable change i.e. it has come down to 2245 and his CD4 came to normal i.e. 1568.

During treatment: Later after 15 days treatment by the Inventor, he got relief from symptoms like Body heat/feverishness, Virtigo, Palpitation, and Loss of Appetite etc. After 2 months treatment all other symptoms subsided. Patient's weight also increased by 2 Kg i.e. 70 Kg to 72 Kg on 29.9.2001.

After treatment: Patient is healthy and happy and his weight increased upto 3 kg. From the above analysis it is proved that these drugs are destroying the virus and hence there is reduction of virus in the body, which is practically proved by you.

The difference in viral load for this patient—

| Viral Load per 1 ml of | | |
|---|---|---|
| plasma Before | Viral Load 1 ml of plasma | viral Destroyed |
| 1 week Treatment | During 3^{rd} month Treatment | within 2 months |
| 6,497 | 2,245 | 4,252 |

### EXAMPLE 5

### Male, 29 years

Duration of treatment: 16.7.2001 to 22.10.2001 (4 months);

Symptoms: Body Heat/feverishness, Dandruff.

Before treatment: The patient before approaching the Inventor his wife was 5th month pregnant and at that time blood report was Reactive HIV. infection. Then he was tested at one private Hospitals Referred by one of the doctor for Western blot test - for HIV report showed HIV-I Reactive & HIV-II Non Reactive. Again on 11.7.2001 he was tested for CD4 & CD8 count -549/2000. Before treatment he was tested at one standard Lab on 23.7.2001 HIV-RNA-PCR-ULTRA QUANT (Viral load) -5,62,000 referred by one government institute Doctor. After 2 months this treatment i.e. as on 25.9.2001, his Viral Load showed remarkable change i.e. it has come down to 52101 and his CD4 came to normal i.e. 459.

During treatment: Later after 15 days treatment by the Inventor he got relief from feverishness . After taking treatment for 2 months he was tested at one standard lab. As on 25.9.2001, his HIV-RNA-PCR- ULTRA QUANT (Viral load) 52101 and his CD4 came to normal i.e.459.

After treatment: Patient is healthy and happy and his weight increased up to 4 kg.

The difference in viral load for this patient—

| Viral Load per | | |
|---|---|---|
| 1 ml of plasma | Viral Load 1 ml of plasma | Destroyed virus |
| 1 week Before Treatment | 2 months After Treatment | within 2 months |
| 562000 | 52101 | 509899 |

### EXAMPLE 6

### Female, 26 years;

Duration of treatment: 24.8.2001 to 25.12.2001 (4 months);

Symptoms: Body Heat/Feverishness, Joint Pains, Headache, Cough, Numbness, Falling of Hair.

Before treatment: The patient's husband was having HIV and hence she got infected through him. The patient before approaching the Inventor was tested at private Hospital by self-Reference—for HIV diagnosis . Elisa test showed HIV I Reactive & HIV-II Non Reactive.

During treatment: Later after 15 days treatment patient got relief from feverishness. On 28.8.2001 during the first week of her treatment she has undergone HIV-RNA-PCR-ULTRA-QUANT and found 18723 and her absolute CD4 count was 474. After taking treatment for 2 months she was tested at one lab as on 16.11.2001, her HIV-RNA-PCR- ULTRA QUANT (Viral load) 3895 and his CD4 came to normal i.e.1003.

After treatment: Patient is healthy and happy.

The difference in viral load for this patient—

| Viral Load | Viral Load | Viral |
|---|---|---|
| During Treatment | During Treatment | Destroyed |
| 1st week | 3rd month | within 3 months |
| 18723 | 3895 | 14828 |

We are presenting few reports of HIV/AIDS Patients who have undergone HIV-RNA-PCR-ULTRA QUANT (Viral Load) test reports, which have shown Astonishing results.

### Virus Destruction per ML of Plasma

| Patient Age, Sex | 1^{st} time Viral Load | 2^{nd} time Viral Load |
|---|---|---|
| 1. 40, Male | HIV-1-10 bands | Western Blot test |
| | Positive | 8 bands Negative |
| | Viral Load - 70,797 | |
| 2. 63, Male | Viral Load - 89,126 | Viral Load - 14,555 |
| 3. 27, Male | Viral Load - 31,018 | Viral Load - 22,759 |
| 4. 56, Male | Viral Load - 6,497 | Viral Load - 2,245 |
| 5. 26, Female | Viral Load - 18,723 | Viral Load - 3,895 |
| 6. 35, Male | Viral Load - 37,889 | Viral Load - <20 |
| 7. 29, Male | Viral Load - 5,62,000 | Viral Load - 52,101 |

For almost all patients result is same in almost all aspects. The viral load result also depends on the behavior of the patient especially avoidance/limitations of intercourse and other restrictions. Thus from the above discussion it must have been very clear that through Viral load test one can know in how much duration of time patient gets negative and also know how much HIV viral is destroyed with in few days/months after the completion of treatment.

Here we are presenting Brief general consent of HIV infected AIDS patient.

### EXAMPLE 7

### 40,Male, Wt: 70 Kg. Merchant Married.

Period of treatment—28-3-97 to 27.7.97.

Patient has come to the inventor after confirmation of his disease from one standard laboratory and referred by concerned Doctor/ Institution after using some medicine for almost 3 years.

Routine Examinations and Assessments: Before commencement of the treatment. General information is elicited from the patient, complete medical history, physical examination, HIV Screening (ELISA), HIV Confirmation (Western Blot) tests, viral load (HIV-RNA-PCR-ULTRA-QUANT) CD4 & CD8 cell counts will be undertaken.

General Management: Consent is obtained from the patient, strict confidentiality of all data collected is maintained to prevent embarrassment to the patient, as HIV infection/AIDS carries a social stigma.

Patient is managed as out patient basis (State II & III & IV). Patient is issued the appropriate drug in systematic manner. Patient/his relative/friend is instructed to know the condition of the patient after 15 days (after 1st dose). A record of the drugs consumed is maintained. Periodic assessments, if any, made by any other physician, who medicates the patient is also maintained.

Patient is instructed to avoid all other forms of medication during this treatment for HIV infection, any other associated illness. He should report to the physician immediately and take appropriate treatment (instructed to take allopathic medicine).

Brief History Before treatment: The patient was suffering from the following symptoms like Body Heat, Loss of Appetite, Stranguria, Pain of Limbs, Giddiness, Palm Cracks, Weight Loss, Body Pains, Decreasing of vision in left eye and fear.

The patient before approaching the Inventor has undergone

Western Blot test on 14.3.97

Elisa test on 31.5.97 at by one doctor (one private Hospital)

Elisa test on 12.7.97 and All the above tests shown HIV-I & HIV-II+VE (positive)

During treatment: The Patient after approaching the Inventor with reports of 8.4.97 (one standard lab) CD4 & CD8 referred by Inventor -213-505 respectively, at one government institute Hospital, On 1.8.97-CD4 & CD8-355-223. On 18.4.98-(one standard lab)-CD4 & CD8-521-1135. (In these tests CD4 increased 213 to 355 to 521).

Follow Up 1: Patient was Issued 15 days medicine Avalmic-I, Avalmic-II, Kaphyog and advised precautions and other diet restrictions and schedule of. medicine.

Follow Up 2: After 15 days treatment we observed lot of improvement in Patient's health condition. Patient has undergone CD4 test one standard lab on 8.4.97 ., CD4 & CD8 referred by Inventor-213-505 respectively at one government institute Hospital. From 2nd day onwards body heat/feverishness subsided, appetite improved. Within 7 days body pains and after 10 days Giddiness subsided. Again issued 15 days medicine Avalmic-I, Avalmic-II and Kaphyog.

Follow Up 3: After 30 days, there was lot of improvement in patients' condition and there was brightness in the face. Again issued 15 days medicine Avalmic-I, Avalmic-II and Kaphyog.

Follow Up 4: After 45 days- Health condition was good and no side effects noticed. Patient was feeling healthy and happy. Later issued 15 days medicine—Avalmic-I, Avalmic-II and Kaphyog.

Follow Up 5: After 60 days- there was increase in the weight up to 2 Kg of the patient. He did not have any associated diseases. Later issued 15 days medicine Avalmic-II, Kaphyog and Medhamruth.

Follow Up 6: After 75 days-Health condition was good and the patient was healthy and happy. Issued 15 days medicine Avalmic-II, Kaphyog and Medhamruth

Follow Up 7: After 90 days- Pain in Limbs reduced. Patient was healthy and happy. There was increase in the weight up to 3 Kg of the patient. Again issued 15 days medicine Avalmic-II, Kaphyog and Medhamruth.

Follow Up 8: After 120 days—all Symptoms subsided without any side effects and vision problem decreased. There was increase in the weight up to 4 Kg. Again issued 15 days medicine—Avalmic-II, Kaphyog and Medhamruth.

Patient has undergone CD4 test on 1.8.97-CD4 & CD8-355-223. Before treatment CD4 was 213 and after treatment his CD4 raised to 355, which, is normal. Hence with the usage of this medicine there was lot of improvement in his absolute CD4 count.

The above patient had taken full course of treatment for four (4) months on Vegetarian based diet only. All accompanying symptoms subsided within 15 days to 30 days giving him great relief mentally and physically.

After treatment: After completion of the treatment health is consistent without any side effects and able to continue his professional happily. He has undergone CD4 test to know his health condition.

On 18.4.98- undergone CD4 test at one standard lab report showed—CD4 & CD8-521-1135. (In these tests CD4 increased 213 to 355 to 521).

Here his absolute CD4 count still increased to 521, which is a remarkable progress. This shows that though the patient stopped the usage of medicine, still his absolute CD4 count has shown remarkable improvement, which shows that immunity has raised in his body.

After treatment, again under gone Absolute CD4 test at one standard lab on 17.2.99—showed CD4 & CD8 632-1142 Respectively. (There is an improvement in CD4 & CD8 after treatment by 419, i.e. 213 to 632).

One Lab report of Western Blot test as on 15.2.99, Reactive for HIV-I, HIV-II-ve, and Specific Band not detected. On 29.10.99—patient was treated at one private lab for—Western Blot. Test showed HIV-II Negative and HIV-II (8 Bonds-ve) (Only P-51 & P-17-2 Bonds+ve). On 6.4.2000, patient again done one private lab for—Western Blot Test showed (.W.B. Test HIV-II Negative and HIV-I (P 17&P51-2 Bonds+ve).

His CD4 & CD8 cell counts reports on 8.4.97-CD4 & CD8-213-505, 1.8.97 CD4 & CD8-355-233,18.4.98 CD4 & CD8 521-1135,17.2.99 CD4 & CD8-632-1142 (Increased to 213 to 355 to 521 to 632) with encouraging result.

VIRAL LOAD TEST for HIV-RNA- PCR-ULTRA- QTY - the patient has undergone a test at one standard lab on 16.12.97 showed per 1 ml of plasma (70797) after 5 months of full treatment. It is evident that the patient must have viral load much higher prior to the initiation of treatment as indicated above i.e. to the tune of ¼ to 1 million of virus in 1 ml of plasma.

After treatment the Patient was free of symptoms. Before treatment his Wife was aborted twice. So he was asked by Doctor to undergo lab test—In the said test, it came to light that the Patient was suffering from HIV infection and after complete treatment his wife was also tested and found to be HIV-ve (Negative).

After treatment his wife was pregnant and she was healthy, and found to be HIV-ve (Negative) on 8.4.2000. She gained weight by 9 Kgs. She gave birth to healthy baby. All three members got negative result. After treatment she was never aborted again.

The patient at present is very healthy and mentally fine and doing his own Profession satisfactorily.

Our Observation: This patient is having viral load in ¼ to 1 million per ml plasma, so HIV-I & II infection turn Negative with in 3 years. Same as above in Western Blot test HIV-I & II all bands turned Negative.

What I claim my invention is:

1. An individual formulation of plants (medicinal plants) of a therapeutically effective amount of Ayurvedic composition 1 (Avalmic-I), comprising a mixture Pulaparni, Vallijam, Palandu, Karanajaka, Chanaka, Sigda, Bharbura and Herbal powder base.

2. An individual formulation of herbal based medicine for a therapeutically effective amount of Ayurvedic composition 2 (Avalmic-I) RASNA, PARADA, PUSHAPAKASEES, SULVA, AMLAKI, and HERBAL POWDER BASE- Quantity Sufficient.

3. An individual formulation of plants (medicinal plants) of a therapeutically effective amount of ayurvedic herbal base composition 3 (Kaphyog) Amlaki, Mahanimba, Tilapushpa, Vajravalli, Ankola, Tulasi, Gandhamula, Amruta Trikatuka.

4. An individual formulation of herbal base medicine for a therapeutically effective amount of ayurvedic composition 4 (Medhamrut) Ananta, Varahakarni, Silaja, Silajit, Talpotali, Mahanimba, Madhuyasti, Chakrangi, Ratanpursha.
